(19) United States
(12) Patent Application Publication
(10) Pub. No.: US 2025/0000446 A1
(43) Pub. Date: Jan. 2, 2025

(54) PREDICTING NEURODEGENERATIVE DISEASES BASED ON SPEECH ANALYSES

(71) Applicant: Genentech, Inc., South San Francisco, CA (US)

(72) Inventors: Laurence KAHN, Tiburon, CA (US); Edmond Huatung TENG, San Mateo, CA (US); Jessica ROBIN, Vancouver, CA (US)

(21) Appl. No.: 18/884,545

(22) Filed: Sep. 13, 2024

Related U.S. Application Data
(63) Continuation of application No. PCT/US2023/064255, filed on Mar. 13, 2023.
(60) Provisional application No. 63/319,650, filed on Mar. 14, 2022.

(51) Int. Cl. A61B 5/00; A61B 6/03; G10L 25/66; G10L 25/93; G16H 50/20

(52) U.S. Cl. CPC A61B 5/4803; A61B 5/4064; A61B 5/4088; A61B 5/7275; G10L 25/66; G10L 25/93; G16H 50/20; A61B 6/037

(57) ABSTRACT

A method implemented by one or more computer device includes receiving speech data associated with a patient and analyzing the speech data to quantify at least one speech variable over a period of time. The at least one speech variable includes a filled-pause variable. The method thus includes determining an estimate of Tau accumulation based on the quantified at least one speech variable.

### CROSS-REFERENCE TO RELATED APPLICATION

This application is a continuation International Application No. PCT/US2023/064255, filed on Mar. 13, 2023, which claims the benefit of and priority to U.S. Provisional Application No. 63/319,650, filed Mar. 14, 2022, the entire content of which is incorporated herein by reference.

### TECHNICAL FIELD

This application relates generally to speech analysis, and, more particularly, to techniques for predicting neurodegenerative diseases based on patient speech analysis.

### BACKGROUND

Identifying and detecting indications of cognitive decline in patients may help to more effectively prevent and treat neurodegenerative diseases, such as Alzheimer's disease (AD) or other forms of dementia. Neurologically, AD may generally include neurofibrillary tangles that are composed of modified Tau protein, and thus the accumulation of Tau within the brain of an AD patient may be proportional to the progression of AD or severity of AD experienced by the AD patient. Specifically, while the accumulation of beta amyloid within the brain of an AD patient may cease in earlier clinical stages of cognitive decline, such as mild cognitive impairment (MCI) or mild neurocognitive disorder (NCD), the accumulation of Tau within the brain of the AD patient continues to increase with the progression of AD. In other words, the total amount of abnormal Tau within the brain of an AD patient may be indicative of AD stage, progression, and/or severity.

Speech may generally include at least some indication of a patient's cognitive abilities. Additionally, with patients having ubiquitous access to personal electronic devices that may be suitable for capturing patient speech, analyses of speech samples for acoustic properties and linguistic properties and content may be readily performed. Thus, patient speech may offer an alternative to invasive procedures and/or intensive clinical and laboratory testing for screening patients for neurodegenerative diseases, such as Alzheimer's disease (AD) or other forms of dementia.

### SUMMARY

Embodiments of the present disclosure are directed toward one or more computing devices, methods, and non-transitory computer-readable media that may be utilized to determine an estimate of Tau accumulation in a patient based on at least a filled-pause quantified speech variable and to detect severity and progression of neurodegenerative disease in the patient. Specifically, in accordance with the presently disclosed embodiments, one or more computing devices may utilize one or more machine-learning models (e.g., a natural-language processing (NLP) model, a transformer-based language model, an automatic speech recognition (ASR) model) to convert raw audio files of patient speech data into a textual transcript and analyze one or more linguistic speech variables and/or one or more acoustic speech variables for determining an estimate of Tau accumulation for a patient to which the patient speech data corresponds. For example, in some embodiments, the one or more computing devices may analyze the textual transcript to calculate one or more of a filled-pause speech variable, a word-frequency speech variable, a vocabulary-diversity speech variable, and so forth that may be then correlated with one or more the standardized uptake value ratio (SUVR) values of one or more specific Tau positron emission tomography (PET) tracers known to indicate severity and progression of AD. Thus, the one or more processing devices may determine an estimate of Tau accumulation based on one or more of the quantified filled-pause speech variable, the word frequency speech variable, the vocabulary diversity speech variable for the corresponding one of the number of patients, and may thereby detect severity and progression of AD in the patient.

In certain embodiments, one or more computing devices may receive speech data associated with a patient. For example, in one embodiment, receiving the speech data may include receiving an audio file including an electronic recording of the patient speaking. In certain embodiments, the one or more computing devices may then analyze the speech data to quantify at least one speech variable over the period of time, in which the at least one speech variable includes a filled-pause variable. For example, in certain embodiments, analyzing the speech data to quantify at least one speech variable may include analyzing the audio file to quantify the at least one speech variable, which includes an acoustic speech variable. In certain embodiments, analyzing the audio file to quantify the filled-pause variable may include analyzing the audio file to identify pauses between words in the speech data, identifying the pauses that qualify as filled pauses, computing a sum of a total number of the pauses between words in the transcript and a total number of the words in the transcript, dividing a number of the filled pauses by the sum, and returning the quantified filled-pause variable.

In certain embodiments, analyzing the speech data to quantify at least one speech variable may further include generating a transcript based on the speech data and analyzing the transcript to quantify the at least one speech variable. In one embodiment, the at least one speech variable may include a linguistic speech variable. In certain embodiments, analyzing the speech data to quantify the filled-pause variable may include generating a transcript based on the speech data, analyzing the transcript to identify pauses between words in the transcript, identifying the pauses that qualify as filled pauses, computing a sum of a total number of the pauses between words in the transcript and a total number of the words in the transcript, dividing a number of the filled pauses by the sum, and returning the quantified filled-pause variable. In certain embodiments, the linguistic speech variable may be determined by generating a transcript based on the speech data and analyzing the transcript to quantify the at least one speech variable. In one embodiment, the at least one speech variable may include a linguistic speech variable.

In certain embodiments, the at least one speech variable may include a word frequency variable. For example, in certain embodiments, the word frequency variable may be determined by computing, for each of the words in the transcript, a frequency score, calculating, based on the frequency scores for each of the words in the transcript, an average frequency score for the transcript and returning the quantified word frequency variable. In certain embodiments, the at least one speech variable may include a vocabulary diversity variable. For example, in certain embodiments, for each of a number of windows in the transcript, and in which each of the windows includes an approximately equal number of contiguous words in the transcript, the vocabulary diversity variable may be determined by computing a number of unique words in the window, computing a total number of words in the window, computing a vocabulary diversity score for the window by dividing the number of unique words by the total number of words, calculating, based on the vocabulary diversity scores for each of the windows in the transcript, an average vocabulary diversity score for the transcript, and returning the quantified vocabulary diversity variable.

In certain embodiments, analyzing the transcript may include analyzing the speech data utilizing one or more natural-language processing (NLP) machine-learning models. In certain embodiments, the one or more computing devices may then determine an estimate of Tau accumulation based on the quantified at least one speech variable. For example, in certain embodiments, determining the estimate of Tau accumulation may include mapping one or more measures of uptake of a positron emission tomography (PET) tracer by Tau present in brains of other subjects to quantified speech variables of the other subjects and estimating, based on the mapping and the one or more quantified speech variables of the patient, an amount of uptake of a PET tracer by Tau predicted to be present in a brain of the patient. For example, in certain embodiments, the one or more measures of uptake of a PET tracer may include one or more measures of uptake of a [¹⁸F]GTP1 Tau PET tracer. an F-18 flortaucipir Tau PET tracer, a (18)F-THK5351 Tau PET tracer, a [¹⁸F]MK-6240 Tau PET tracer, a RO-948 Tau PET tracer. a PI-2014 Tau PET tracer, a PI-2620 Tau PET tracer, or a T-808 Tau PET tracer.

In certain embodiments, the one or more computing devices may classify the patient, based on the estimate of Tau accumulation, as having a neurodegenerative disease. For example, in one embodiment, the neurodegenerative disease may include Alzheimer's disease (AD). In certain embodiments, the one or more computing devices may determine, based on identified changes in the at least one speech variable over the period of time, a rate of progression of the neurodegenerative disease. For example, in certain embodiments, the one or more computing devices may predict, based on the rate of progression of the neurodegenerative disease, a change in performance of the patient on a clinical assessment selected from the group consisting of a Mini Mental State Examination, a Clinical Dementia Rating Scale interview, a Clinical Dementia Rating-Sum of Boxes interview, a Alzheimer's Disease Assessment Scale—Cognitive Subscale battery of tests, an Alzheimer's disease Cooperative Study Group—Activities of Daily Living Inventory, a Neuropsychiatric Inventory, a Caregiver Global Impression Scale for Alzheimer's Disease, an Instrumental Activities of Daily Living Scale, and an Amsterdam Activities of Daily Living Questionnaire. In certain embodiments, the one or more computing devices may determine the rate of progression of neurodegenerative disease based on a treatment regimen for the patient. In certain embodiments, the one or more computing devices may then transmit a notification regarding the estimate of Tau accumulation to a computing device associated with a clinician. In certain embodiments, the one or more computing devices may also transmit a notification regarding the estimate of Tau accumulation to an electronic device associated with the patient.

In certain embodiments, the one or more computing devices may, in response to determining the estimate of Tau accumulation, generate a recommendation for administration of a therapeutic agent consisting of at least one compound selected from a group consisting of compounds against oxidative stress, anti-apoptotic compounds, metal chelators, inhibitors of DNA repair, 3-amino-1-propanesulfonic acid (3APS), 1,3-propanedisulfonate (1,3PDS), secretase activators, beta-and gamma-secretase inhibitors, tau proteins, anti-Tau antibodies, anti-Tau agents, neurotransmitters, beta-sheet breakers, anti-inflammatory molecules, an atypical antipsychotic, a cholinesterase inhibitor, other drugs, and nutritive supplements, a therapeutic agent selected from the group consisting of: a symptomatic medication, a neurological drug, a corticosteroid, an antibiotic, an antiviral agent, an anti-Tau antibody, a Tau inhibitor, an anti-amyloid beta antibody, an beta-amyloid aggregation inhibitor, a therapeutic agent that binds to a target, an anti-BACE1 antibody, a BACE1 inhibitor, a cholinesterase inhibitor, an NMDA receptor antagonist, a monoamine depletory, an ergoloid mesylate, an anticholinergic antiparkinsonism agent, a dopaminergic antiparkinsonism agent, a tetrabenazine, an anti-inflammatory agent, a hormone, a vitamin, a dimebolin, a homotaurine, a serotonin receptor activity modulator, an interferon, and a glucocorticoid.

### BRIEF DESCRIPTION OF THE DRAWINGS

FIG. 1 illustrates an example embodiment of a telehealth service environment that may be utilized to determine an estimate of Tau accumulation in a patient based on one or more filled-pause quantified speech variables and to detect severity and progression of neurodegenerative disease in the patient.

FIG. 2 illustrates a table diagram of cross-sectional correlations between whole cortical grey [¹⁸F]GTP1 SUVR and global clinical scores.

FIG. 3 illustrates a plot diagram of cross-sectional correlations between whole cortical grey [¹⁸F]GTP1 SUVR and speech variables.

FIG. 4A illustrates a plot diagrams of cross-sectional correlations between [¹⁸F]GTP1 SUVR and filled-pauses generalize across regions of interests (ROIs) of a patient's brain.

FIG. 4B illustrates a plot diagrams of baseline filled-pauses correlated with increases in [¹⁸F]GTP1 SUVR over 18 months.

FIG. 5A illustrates a flow diagram for determining an estimate of Tau accumulation in a patient based on at least a filled-pause quantified speech variable and detecting severity and progression of neurodegenerative disease in the patient.

FIG. 5B illustrates a flow diagram for determining an estimate of Tau accumulation in a patient based on at least a word frequency quantified speech variables and detecting severity and progression of neurodegenerative disease in the patient.

FIG. 5C illustrates a flow diagram for determining an estimate of Tau accumulation in a patient based on at least a vocabulary diversity quantified speech variables and detecting severity and progression of neurodegenerative disease in the patient.

FIG. 6 illustrates an example computing system.

FIG. 7 illustrates a diagram of an example artificial intelligence (AI) architecture included as part of the example computing system of FIG. 6.

### DESCRIPTION OF EXAMPLE EMBODIMENTS

Identifying and detecting indications of cognitive decline in patients may help to more effectively prevent and treat neurodegenerative diseases, such as Alzheimer's disease (AD) or other forms of dementia. Neurologically, AD may generally include neurofibrillary tangles that are composed of modified Tau protein, and thus the accumulation of Tau within the brain of an AD patient may be proportional to the progression of AD or severity of AD experienced by the AD patient. Specifically, while the accumulation of beta amyloid within the brain of an AD patient may cease in earlier clinical stages of cognitive decline, such as mild cognitive impairment (MCI) or mild neurocognitive disorder (NCD), the accumulation of Tau within the brain of the AD patient continues to increase with the progression of AD. In other words, the total amount of abnormal Tau within the brain of an AD patient may be indicative of AD stage, progression, and/or severity.

Speech may generally include at least some indication of a patient's cognitive abilities. Additionally, with patients having ubiquitous access to personal electronic devices that may be suitable for capturing patient speech, analyses of speech samples for acoustic properties and linguistic properties and content may be readily performed. Thus, patient speech may offer an alternative to invasive procedures and/or intensive clinical and laboratory testing for screening patients for neurodegenerative diseases, such as Alzheimer's disease (AD) or other forms of dementia.

Accordingly, the present disclosure are directed toward one or more computing devices, methods, and non-transitory computer-readable media that may be utilized to determine an estimate of Tau accumulation in a patient based on at least a filled-pause quantified speech variable and to detect severity and progression of neurodegenerative disease in the patient. Specifically, in accordance with the presently disclosed embodiments, one or more computing devices may utilize one or more machine-learning models (e.g., a natural-language processing (NLP) model, a transformer-based language model, an automatic speech recognition (ASR) model) to convert raw audio files of patient speech data into a textual transcript and analyze one or more linguistic speech variables and/or one or more acoustic speech variables for determining an estimate of Tau accumulation for a patient to which the patient speech data corresponds. For example, in some embodiments, the one or more computing devices may analyze the textual transcript to calculate one or more of a filled-pause speech variable, a word-frequency speech variable, a vocabulary-diversity speech variable, and so forth that may be then correlated with one or more the SUVR values of one or more specific Tau PET tracers known to indicate severity and progression of AD. Thus, the one or more processing devices may determine an estimate of Tau accumulation based on one or more of the quantified filled-pause speech variable. the word frequency speech variable, the vocabulary diversity speech variable for the corresponding one of the number of patients, and may thereby detect severity and progression of AD in the patient.

As Will Be Further Described Herein with Respect to Therapies or Treatments:

Therapeutic agents may include neuron-transmission enhancers, psychotherapeutic drugs, acetylcholine esterase inhibitors, calcium-channel blockers, biogenic amines, benzodiazepine tranquillizers, acetylcholine synthesis, storage or release enhancers, acetylcholine postsynaptic receptor agonists, monoamine oxidase-A or -B inhibitors, N-methyl-D-aspartate glutamate receptor antagonists. non-steroidal anti-inflammatory drugs, antioxidants, or serotonergic receptor antagonists. In particular, the therapeutic agent may comprise at least one compound selected from compounds against oxidative stress, anti-apoptotic compounds, metal chelators, inhibitors of DNA repair such as pirenzepine and metabolites, 3-amino-1-propanesulfonic acid (3APS), 1,3-propanedisulfonate (1,3PDS), secretase activators, beta-and gamma-secretase inhibitors, tau proteins, anti-Tau antibodies or anti-Tau agents, neurotransmitters, beta-sheet breakers, anti-inflammatory molecules, “atypical antipsychotics” such as, for example clozapine, ziprasidone, risperidone, aripiprazole or olanzapine or cholinesterase inhibitors (ChEIs) such as tacrine, rivastigmine, donepezil, and/or galantamine and other drugs or nutritive supplements such as, for example, vitamin B 12, cysteine, a precursor of acetylcholine, lecithin, choline, Ginkgo biloba, acyetyl-L-carnitine, idebenone, propentofylline, and/or a xanthine derivative.

In some embodiments, the therapeutic agent is a Tau inhibitor. Non-limiting examples of Tau inhibitors include methylthioninium, LMTX (also known as leuco-methylthioninium or Trx-0237; TauRx Therapeutics Ltd.), Rember™ (methylene blue or methylthioninium chloride [MTC]; Trx-0014; TauRx Therapeutics Ltd), PBT2 (Prana Biotechnology), and PTI-51-CH3 (TauPro™; ProteoTech).

In some embodiments, the therapeutic agent is an anti-Tau antibody. “Anti-Tau immunoglobulin,” “anti-Tau antibody” and “antibody that binds Tau” are used interchangeably herein, and refer to an antibody that is capable of binding Tau (e.g., human Tau) with sufficient affinity such that the antibody is useful as a diagnostic and/or therapeutic agent in targeting Tau. In some embodiments, the extent of binding of an anti-Tau antibody to an unrelated, non-Tau protein is less than about 10% of the binding of the antibody to Tau as measured, e.g., by a radioimmunoassay (RIA). In certain embodiments, an antibody that binds to Tau has a dissociation constant (KD) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M. e.g., from 10⁻⁹ M to 10⁻¹³ M). In certain embodiments, an anti-Tau antibody binds to an epitope of Tau that is conserved among Tau from different species. In some cases, the antibody binds monomeric Tau, oligomeric Tau, and/or phosphorylated Tau. In some embodiments, the anti-Tau antibody binds to monomeric Tau, oligomeric Tau, non-phosphorylated Tau, and phosphorylated Tau with comparable affinities, such as with affinities that differ by no more than 50-fold from one another. In some embodiments, an antibody that binds monomeric Tau, oligomeric Tau, non-phosphory lated Tau, and phosphorylated Tau is referred to as a “pan-Tau antibody.” In some embodiments, the anti-Tau antibody binds to an N-terminal region of Tau, for example, an epitope within residues 2 to 24, such as an epitope within/spanning residues 6 to 23. In a specific embodiment, the anti-Tau antibody is semorinemab.

In some embodiments, the anti-Tau antibody is one or more selected from the group consisting of a different N-terminal binder, a mid-domain binder, and a fibrillar Tau binder. Non-limiting examples of other anti-Tau antibodies include BIIB092 or BMS-986168 (Biogen, Bristol-Myers Squibb); APN-mAb005 (Aprinoia Therapeutics/Samsung Biologics), BIIB076 (Biogen/Eisai), ABBV-8E12 or C2N-8E12 (AbbVie, C2N Diagnostics, LLC); an antibody disclosed in WO2012049570, WO2014028777, WO2014165271, WO2014100600, WO2015200806, U.S. Pat. Nos. 8,980,270, or 8,980,271; E2814 (Eisai), Gosuranemab (Biogen), Tilavonemab (Abbvie), and Zagotenemab (Lilly).

In some embodiments, the therapeutic agent is an anti-Tau agent. Non limiting examples include BIIB080 (Biogen/Ionis), LY3372689 (Lilly), PNT001 (Pinteon Therapeutics), OLX-07010 (Oligomerix, Inc.), TRx-0237/LMTX (TauRx), JNJ-63733657 (Janssen), Tau siRNA (Lilly/Dicerna), and PSY-02 (Psy Therapeutics).

In some embodiments, the therapeutic agent is at least one compound for treating AD, selected from the group consisting of GV-971 (Green Valley), CT1812 (Cognition Therapeutics), ATH-1017 (Athira Pharma), COR388 (Cortexyme), simufilam (Cassava), semaglutide (Novo Nordisk), Blarcamesine (Anavex Life Sciences), AR1001 (AriBio), Nilotinib BE (KeifeRx/Life Molecular Imaging/Sun Pharma), ALZ-801 (Alzheon), AL003 (Alector/AbbVie), Lomecel-B (Longeveron), UB-311 (Vaxxinity), XPro1595/Pegipanermin (INmune Bio), NLY-01 (D&D Biotech), Varoglutamstat/PQ912 (Vivoryon/Nordic/Simcere), Canakinumab (Novartis), Obicetrapib (New Amsterdam Pharma), AADvac1 (Axon Neuroscience), ANVS-401/Posiphen (Annovis Bio), TB006 (TureBinding), BI 474121 (Boehringer Ingelheim), NuCerin (Shaperon/Kukjeon), (Alzinova) ALZ-101, NNI-362 (Neuronascent), MK-1942 (Merck), E2511 (Eisai), IGC-ADI (India Globalization Capital), AL001 (Alzamend Neuro), AL002 (Alzamend Neuro), AL101 (Alector/GSK), MW-151 (ImmunoChem Therapeutics), DNL-788/SAR443820 (Denali/Sanofi), ALN-APP (Alnylam/Regeneron), E2F4DN (Tetraneuron), EmtinB (NeuroScientific Biopharma), NIT-001 (Neurostech), ACD679 (AlzeCure Pharma), ACD680 (AlzeCure Pharma), YDC-103 (YD Global Life Science), BMD-001 (Biorchestra), STL-101 (Stellate Therapeutics), AV-1959R (Nuravax), AV1959D (Nuravax), AV1980R (Nuravax), Duvax (Nuravax), Dapansutrile (Olatec Therapeutics), LX1001 (Cornell University), BDNF (UC San Diego), ST-501 (Biogen), AMT-240 (uniQure), SOL-410 (Sola), SOL-258 (Sola), AAVhmAb, SHP-231 (Shape), SHP-232 (Shape), TEL-01 (Telocyte), GT-0007X (Gene Therapy).

In some embodiments, the therapeutic agent is a general misfolding inhibitor, such as NPT088 (NeuroPhage Pharmaceuticals).

In some embodiments, the therapeutic agent is a neurological drug. Neurological drugs include, but are not limited to, an antibody or other binding molecule (including, but not limited to a small molecule, a peptide, an aptamer, or other protein binder) that specifically binds to a target selected from: beta secretase, presenilin, amyloid precursor protein or portions thereof, amyloid beta peptide or oligomers or fibrils thereof, death receptor 6 (DR6), receptor for advanced glycation endproducts (RAGE), parkin, and huntingtin; an NMDA receptor antagonist (i.e., memantine), a monoamine depletor (i.e., tetrabenazine); an ergoloid mesylate; an anticholinergic antiparkinsonism agent (i.e., procyclidine, diphenhydramine, trihexylphenidyl, benztropine, biperiden and trihexyphenidyl); a dopaminergic antiparkinsonism agent (i.e., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (i.e., indomethicin and other compounds listed above); a hormone (i.e., estrogen, progesterone and leuprolide); a vitamin (i.e., folate and nicotinamide); a dimebolin; a homotaurine (i.e., 3-aminopropanesulfonic acid; 3APS); a serotonin receptor activity modulator (i.e., xaliproden); an interferon, and a glucocorticoid or corticosteroid. The term “corticosteroid” includes, but is not limited to, fluticasone (including fluticasone propionate (FP)), beclometasone, budesonide, ciclesonide, mometasone, flunisolide, betamethasone and triamcinolone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, and triamcinolone acetonide.

In certain particular embodiments, the therapeutic agent is one or more selected from the group of a corticosteroid, an antibiotic, an antiviral agent, an anti-Tau antibody, a Tau inhibitor, an anti-amyloid beta antibody, an beta-amyloid aggregation inhibitor, an anti-BACE1 antibody, a BACE1 inhibitor; a therapeutic agent that specifically binds a target; a cholinesterase inhibitor; an NMDA receptor antagonist; a monoamine depletor; an ergoloid mesylate; an anticholinergic antiparkinsonism agent; a dopaminergic antiparkinsonism agent; a tetrabenazine; an anti-inflammatory agent; a hormone; a vitamin; a dimebolin; a homotaurine; a serotonin receptor activity modulator; an interferon, and a glucocorticoid.

Non-limiting examples of anti-Abeta antibodies include crenezumab, solanezumab (Lilly), bapineuzumab, aducanumab, gantenerumab, donanemab (Lilly), LY3372993 (Lilly), ACU193 (Acumen Pharmaceuticals). SHR-1707 (Hengrui USA/Atridia), ALZ-201 (Alzinova), PMN-310 (ProMIS neurosciences), and lecanemab (BAN-2401; Biogen, Eisai Co., Ltd.). Non-limiting exemplary beta-amyloid aggregation inhibitors include ELND-005 (also referred to as AZD-103 or scyllo-inositol), tramiprosate, and PTI-80 (Exebryl-1®; ProteoTech). Non-limiting examples of BACE inhibitors include E-2609 (Biogen, Eisai Co., Ltd.), AZD3293 (also known as LY3314814; AstraZeneca, Eli Lilly & Co.), MK-8931 (verubecestat), and JNJ-54861911 (Janssen, Shionogi Pharma).

In some embodiments, the therapeutic agent is an “atypical antipsychotic,” such as, e.g., clozapine, ziprasidone, risperidone, aripiprazole or olanzapine for the treatment of positive and negative psychotic symptoms including hallucinations, delusions, thought disorders (manifested by marked incoherence, derailment, tangentiality), and bizarre or disorganized behavior, as well as anhedonia, flattened affect, apathy, and social withdrawal.

Other therapeutic agents, in some embodiments, include, e.g., therapeutic agents discussed in WO 2004/058258 (see especially pages 16 and 17), including therapeutic drug targets (page 36-39), alkanesulfonic acids and alkanolsulfuric acid (pages 39-51), cholinesterase inhibitors (pages 51-56), NMDA receptor antagonists (pages 56-58), estrogens (pages 58-59), non-steroidal anti-inflammatory drugs (pages 60-61), antioxidants (pages 61-62), peroxisome proliferators-activated receptors (PPAR) agonists (pages 63-67), cholesterol-lowering agents (pages 68-75); amyloid inhibitors (pages 75-77), amyloid formation inhibitors (pages 77-78), metal chelators (pages 78-79), anti-psychotics and anti-depressants (pages 80-82), nutritional supplements (pages 83-89) and compounds increasing the availability of biologically active substances in the brain (see pages 89-93) and prodrugs (pages 93 and 94), which document is incorporated herein by reference. but especially the compounds mentioned on the pages indicated above.

As Will be Further Described Herein with Respect to Indications of Tau Accumulation:

The terms “tau pathology,” “tauopathy,” “tau protein-associated disease,” or “tau-associated disease” are used interchangeably herein. and refer to a group of diseases and disorders caused by or associated with Tau aggregates in the extracellular space of a patient's brain, including those caused by or associated with the formation of neurofibrillary or neuropil threads. Such diseases include, but are not limited to, neurological disorders, such as AD, and diseases or conditions characterized by a loss of cognitive capacity. Non-limiting examples of tau pathologies include amyotrophic lateral sclerosis. Parkinson's disease, Creutzfeldt-Jacob disease, dementia pugilistica, Down's syndrome, Gerstmann-Sträussler-Scheinker disease, inclusion-body myositis, prion protein cerebral amyloid angiopathy, traumatic brain injury, amyotrophic lateral sclerosis/parkinsonism-dementia complex of Guam, non-Guamanian motor neuron disease with neurofibrillary tangles, argyrophilic grain dementia, corticobasal degeneration, diffuse neurofibrillary tangles with calcification, frontotemporal dementia, frontotemporal dementia with parkinsonism linked to chromosome 17, Hallevorden-Spatz disease, multiple system atrophy, Niemann-Pick disease type C, pallido-ponto-nigral degeneration, Pick's disease, progressive subcortical gliosis, progressive supranuclear palsy, subacute sclerosing panencephalitis, tangle only dementia, postencephalitic parkinsonism, and myotonic dystrophy. In some embodiments, the tauopathy is progressive supranuclear palsy.

As Will be Further Described Herein with Respect to Measurements of Alzheimer's Disease Severity and Progression:

The Mini Mental State Examination (“MMSE”) is a brief clinical cognitive examination commonly used to screen for dementia and other cognitive deficits (Folstein et al. J Psychiatr Res 1975;12:189-98). The MMSE provides a total score of 0-30. Scores of 26 and lower are generally considered to indicate a deficit. The lower the numerical score on the MMSE, the greater the tested patient's deficit or impairment relative to another individual with a higher score. An increase in MMSE score may be indicative of improvement in the patient's condition, whereas a decrease in MMSE score may denote worsening in the patient's condition. In some embodiments, a stable MMSE score may be indicative of a slowing, delay, or halt of the progression of AD, or a lack of appearance of new clinical, functional, or cognitive symptoms or impairments, or an overall stabilization of disease.

The Clinical Dementia Rating Scale (“CDR”) (Morris Neurology 1993;43:2412-4) is a semi structured interview that yields five degrees of impairment in performance for each of six categories of cognitively based functioning: memory, orientation, judgment and problem solving, community affairs, home and hobbies, and personal care. The CDR was originally designed with a global score: 0—no dementia; 0.5—questionable dementia, 1—mild dementia, 2—moderate dementia, 3—severe dementia.

A complete CDR-SB score is based on the sum of the scores across all 6 boxes. Subscores can be obtained for each of the boxes or components individually as well, e.g., CDR/Memory or CDR/Judgment and Problem solving. As used herein, a “decline in CDR-SB performance” or an “increase in CDR-SB score” indicates a worsening in the patient's condition and may reflect progression of AD.

The term “CDR-SB” refers to the Clinical Dementia Rating-Sum of Boxes, which provides a score between 0 and 18 (O'Bryant et al., 2008, Arch Neurol 65: 1091-1095). CDR-SB score is based on semi-structured interviews of patients and caregiver informants, and yields five degrees of impairment in performance for each of six categories of cognitively-based functioning: memory, orientation, judgment/problem solving, community affairs, home and hobbies, and personal care. The test is administered to both the patient and the caregiver and each component (or each “box”) is scored on a scale of 0 to 3 (the five degrees are 0, 0.5, 1, 2, and 3). The sum of the score for the six categories is the CDR-SB score. A decrease in CDR-SB score may be indicative of improvement in the patient's condition, whereas an increase in CDR-SB score may be indicative of worsening of the patient's condition. In some embodiments, a stable CDR-SB score may be indicative of a slowing, delay, or halt of the progression of AD, or a lack of appearance of new clinical, functional, or cognitive symptoms or impairments, or an overall stabilization of disease.

The Alzheimer's Disease Assessment Scale-Cognitive Subscale (“ADAS Cog”) is a frequently used scale to assess cognition in clinical trials for mild-to-moderate AD (Rozzini et al. Int J Geriatr Psychiatry 2007;22:1217-22.; Connor and Sabbagh, J Alzheimers Dis. 2008;15:461-4: Ihl et al. Int J Geriatr Psychiatry 2012;27:15-21). The ADAS-Cog is an examiner-administered battery that assesses multiple cognitive domains, including memory, comprehension, praxis, orientation, and spontaneous speech (Rosen et al. 1984, Am J Psychiatr 141:1356-64; Mohs et al. 1997, Alzheimer Dis Assoc Disord 11(S2):S13-S21). The ADAS-Cog is a standard primary endpoint in AD treatment trials (Mani 2004, Stat Med 23:305-14). The higher the numerical score on the ADAS-Cog, the greater the tested patient's deficit or impairment relative to another individual with a lower score. The ADAS-Cog may be used to assess whether a treatment for AD is therapeutically effective. An increase in ADAS-Cog score is indicative of worsening in the patient's condition, whereas a decrease in ADAS-Cog score denotes improvement in the patient's condition. In some embodiments, a stable ADAS-Cog score may be indicative of a slowing, delay, or halt of the progression of AD, or a lack of appearance of new clinical or cognitive symptoms or impairments, or an overall stabilization of disease.

The ADAS-Cog12 is the 70-point version of the ADAS-Cog plus a 10-point Delayed Word Recall item assessing recall of a learned word list. The ADAS-Cog11 is another version, with a range from 0-70. Other ADAS-Cog scales include the ADAS-Cog13 and ADAS-Cog14.

A decrease in ADAS-Cog11 score may be indicative of improvement in the patient's condition, whereas an increase in ADAS-Cog11 score may be indicative of worsening of the patient's condition. In some embodiments, a stable ADAS-Cog11 score may be indicative of a slowing, delay, or halt of the progression of AD, or a reduction in the progression of clinical or cognitive decline, or a lack of appearance of new clinical or cognitive symptoms or impairments, or an overall stabilization of disease.

The component subtests of the ADAS-Cog11 can be grouped into three cognitive domains: memory, language, and praxis (Verma et al. Alzheimer's Research & Therapy 2015). This “breakdown” can improve sensitivity in measuring decline in cognitive capacity, e.g., when focused in the mild-to-moderate AD stage (Verma, 2015). Thus ADAS-Cog11 scores can be analyzed for changes on each of three cognitive domains: a memory domain, a language domain, and a praxis domain. A memory domain value of an ADAS-Cog11 score may be referred to herein as an “ADAS-Cog11 memory domain score” or simply “memory domain.” Slowing memory decline may refer to reducing rate of loss in memory capacity and/faculty, retaining memory, and/or reducing memory loss. Slowing memory decline can be evidenced, e.g., by smaller (or less negative) scores on the ADAS-Cog11 memory domain.

Similarly, a language domain value of an ADAS-Cog11 score may be referred to herein as an “ADAS-Cog11 language domain score” or simply “language domain; ” and a praxis domain value of an ADAS-Cog11 score may be referred to herein as an “ADAS-Cog11 praxis domain score” or simply “praxis domain.” Praxis can refer to the planning and/or execution of simple tasks and/or praxis can refer to the ability to conceptualize, plan, and execute a complex sequence of motor actions, as well as copy drawings or three-dimensional constructions, and following commands.

The memory domain score is further divided into components including scores reflecting a subject's ability to recognize and/or recall words, thereby assessing capabilities in “word recognition” or “word recall.” A word recognition assessment of an ADAS-Cog11 memory domain score may be referred to herein as an “ADAS-Cog11 word recognition score” or simply “word recognition score.” For example, equivalent alternate forms of subtests for word recall and word recognition can be used in successive test administrations for a given patient. Slowing memory decline can be evidenced, e.g., by smaller (or less negative) scores on the word recognition component of the ADAS-Cog11 memory domain.

The Alzheimer's disease Cooperative Study Group—Activities of Daily Living Inventory or the Alzheimer's disease Cooperative Study Group—Activities of Daily Living Scale (“ADCS-ADL;” Galasko et al. Alzheimer Dis Assoc Disord 1997; 11(Suppl 2):S33-9) is the most widely used scale for assessing functional outcomes in patients with AD (Vellas et al. Lancet Neurol. 2008;7:436-50). Scores range from 0 to 78, with higher scores indicating better ADL function. The ADCS-ADL is administered to caregivers and covers both basic ADL (e.g., eating and toileting) and more complex ADL or instrumental ADL (e.g., using the telephone, managing finances, preparing a meal) (Galasko et al. Alzheimer Disease and Associated Disorders, 1997 11(Suppl2), S33-S39).

The Neuropsychiatric Inventory (“NPI”) (Cummings et al. Neurology 1994; 44:2308-14) is a widely used scale that assesses the behavioral symptoms in AD, including their frequency, severity, and associated distress. Individual symptom scores range from 0 to 12 and total NPI scores range from 0 to 144. NPI is administered to caregivers, and refers to the behavior of the patient over the preceding month.

The Caregiver Global Impression Scales for Alzheimer's Disease (“CaGI-Alz”) is a novel scale used in clinical studies described herein, and is comprised of four items to assess the caregiver's perception of the patient's change in disease severity. All items are rated on a 7-point Likert type scale from 1 (very much improved since treatment started/previous CaGI Alz assessment) to 7 (very much worsened since treatment started/previous CaGI Alz assessment).

The term “iADL” refers to the Instrumental Activities of Daily Living scale (Lawton, M. P., and Brody, E. M., 1969, Gerontologist 9: 179-186). This scale measures the ability to perform typical daily activities such as housekeeping, laundry, operating a telephone, shopping, preparing meals, etc. The lower the score, the more impaired the individual is in conducting activities of daily living.

Another scale that may be used is the “Amsterdam Activities of Daily Living Questionnaire (A-IADL-Q).”

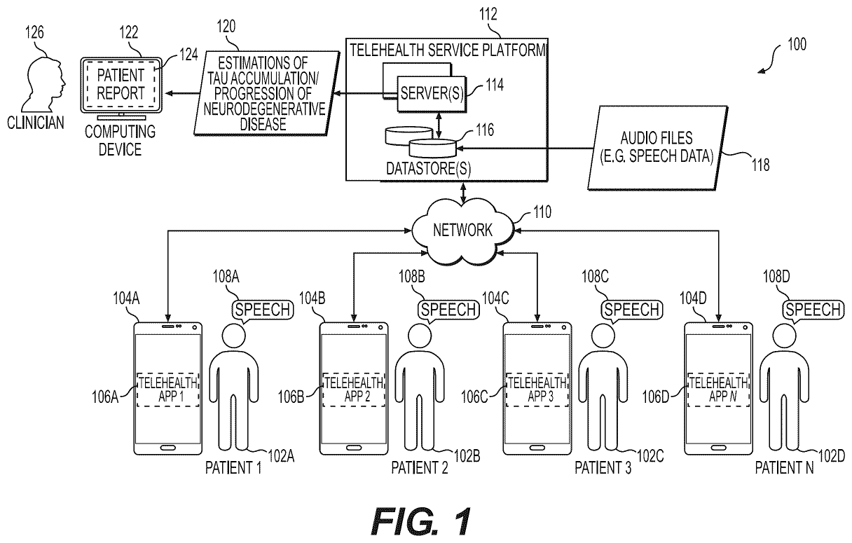

FIG. 1 illustrates an example embodiment of a telehealth service environment 100 that may be utilized to determine an estimate of Tau accumulation in a patient based on at least a filled-pause quantified speech variable and to detect severity and progression of neurodegenerative disease in the patient, in accordance with the presently disclosed embodiments. As depicted, telehealth service environment 100 may include a number of patients 102A, 102B, 102C, and 102D each associated with respective electronic devices 104A, 104B, 104C, and 104D that may be suitable for allowing the number of patients 102A, 102B, 102C, and 102D to launch and engage respective telehealth applications 106A (e.g., “Telehealth App 1”), 106B (e.g., “Telehealth App 2”), 106C (e.g., “Telehealth App 3”), and 106D (e.g., “Telehealth App N”). Specifically, as depicted by FIG. 1, the respective electronic devices 104A, 104B, 104C, and 104D may be coupled to a telehealth service platform 112 via one or more communication network(s) 110. In certain embodiments, the telehealth service platform 112 may include, for example, a cloud-based computing architecture suitable for hosting and servicing the telehealth applications 106A (e.g., “Telehealth App 1”), 106B (e.g., “Telehealth App 2”), 106C (e.g., “Telehealth App 3”), and 106D (e.g., “Telehealth App N”) executing on the respective electronic devices 104A, 104B, 104C, and 104D. For example, in one embodiment, the telehealth service platform 112 may include a Platform as a Service (PaaS) architecture, a Software as a Service (SaaS) architecture, an Infrastructure as a Service (IaaS) architecture, a Compute as a Service (CaaS) architecture, a Data as a Service (DaaS) architecture, a Database as a Service (DBaaS) architecture, or other similar cloud-based computing architecture (e.g., “X” as a Service (XaaS)).

In certain embodiments, as further depicted by FIG. 1. the telehealth service platform 112 may include one or more processing devices 114 (e.g., servers) and one or more data stores 116. For example, in some embodiments, the one or more processing devices 114 (e.g., servers) may include one or more a general purpose processors, a graphic processing units (GPUs), application-specific integrated circuits (ASICs), systems-on-chip (SoCs), microcontrollers, field-programmable gate arrays (FPGAs), central processing units (CPUs), application processors (APs), visual processing units (VPUs), neural processing units (NPUs), neural decision processors (NDPs), deep learning processors (DLPs), tensor processing units (TPUs), neuromorphic processing units (NPUs), or any of various other processing device(s) or accelerators that may be suitable for providing processing and/or computing support for the telehealth applications 106A (e.g., “Telehealth App 1”), 106B (e.g., “Telehealth App 2”), 106C (e.g., “Telehealth App 3”), and 106D (e.g., “Telehealth App N”). Similarly, the data stores 116 may include, for example, one or more internal databases that may be utilized to store information (e.g., audio files of patient speech data 118) associated with the number of patients 102A, 102B, 102C, and 102D.

In certain embodiments, as previously noted, the telehealth service platform 112 may be a hosting and servicing platform for the telehealth applications 106A (e.g., “Telehealth App 1”), 106B (e.g., “Telehealth App 2”), 106C (e.g., “Telehealth App 3”), and 106D (e.g., “Telehealth App N”) executing on the respective electronic devices 104A, 104B, 104C, and 104D. For example, in some embodiments, the telehealth applications 106A (e.g., “Telehealth App 1”), 106B (e.g., “Telehealth App 2”), 106C (e.g., “Telehealth App 3”), and 106D (e.g., “Telehealth App N”) may each include, for example, telehealth mobile applications (e.g., mobile apps) that may be utilized to allow the number of patients 102A, 102B, 102C, and 102D to access health care services and medical care services remotely and/or to engage with one or more patient-selected clinicians (e.g., clinicians 126) as part of an on-demand health care service.

In certain embodiments, one or more of the number of patients 102A, 102B, 102C, and 102D may include one or more patients having AD, one or more patients suspected of having AD, and/or one or more patients predisposed to developing AD. Thus, as further depicted by FIG. 1, in certain embodiments, one or more of the number of patients 102A, 102B, 102C, and 102D may undergo a speech-based assessment utilized to determine an estimate of Tau accumulation in one or more of the number of patients 102A, 102B, 102C, and 102D utilizing at least a filled-pause quantified speech variable. For example, in certain embodiments, one or more of the number of patients 102A, 102B, 102C, and 102D may input speech 108A, 108B, 108C, 108D utilizing the telehealth applications 106A (e.g., “Telehealth App 1”), 106B (e.g., “Telehealth App 2”), 106C (e.g., “Telehealth App 3”), and 106D (e.g., “Telehealth App N”) executing on the respective electronic devices 104A, 104B, 104C, and 104D. For example, in some embodiments, the inputted speech 108A, 108B, 108C, 108D may include, for example, an electronic recording of the number of patients 102A, 102B, 102C, and 102D speaking. In certain embodiments, the inputted speech 108A, 108B, 108C, 108D may be performed in response to, for example, one or more requests provided by the telehealth service platform 112 to one or more of the number of patients 102A, 102B, 102C, and 102D via the telehealth applications 106A (e.g., “Telehealth App 1”), 106B (e.g., “Telehealth App 2”), 106C (e.g., “Telehealth App 3”), and 106D (e.g., “Telehealth App N”). In other embodiments, one or more of the number of patients 102A, 102B, 102C, and 102D may record the inputted speech 108A, 108B, 108C, 108D utilizing one or more microphones of the respective electronic devices 104A, 104B, 104C, and 104D without first being prompted via the telehealth applications 106A (e.g., “Telehealth App 1”), 106B (e.g., “Telehealth App 2”), 106C (e.g., “Telehealth App 3”), and 106D (e.g., “Telehealth App N”).

For example, in some embodiments, as part of the speech-based assessment, the telehealth service platform 112 may generate and provide one or more speech-based tasks that prompt one or more of the number of patients 102A, 102B, 102C, and 102D to produce speech and record by way of one or more microphones of the respective electronic devices 104A, 104B, 104C, and 104D. In one embodiment, the speech-based assessment may include, for example, a description of an image that may be displayed via the telehealth applications 106A, 106B, 106C, and 106D, a reading of a book passage that may be presented via the telehealth applications 106A, 106B, 106C, and 106D, a series of question-response tasks that may be presented via the telehealth applications 106A, 106B, 106C, and 106D, or other speech-based assessments in accordance with medical-grade neuropsychological speech and language assessments. In certain embodiments, the speech-based assessment may be performed at different moments in time over some given period of time. For example, in some embodiments, the speech-based assessment may be performed at an initial date and then again, for example, at one or more dates selected from the group comprising: approximately 0.25 months, 0.5months, 0.75 months, 1 month, 3 months, 6 months, 9 months, 12 months, 15 months, 18months, 21 months, 24 months, 27 months, 30 months, 33 months, and/or 36 months from the initial date.

In certain embodiments, upon one or more of the number of patients 102A, 102B, 102C, and 102D completing the speech-based assessment, one or more of the respective electronic devices 104A, 104B, 104C, and 104D may then transmit one or more audio files of patient speech data 118 to the telehealth service platform 112. In certain embodiments, the one or more audio files of patient speech data 118 may be stored to the one or more data stores 116 of the telehealth service platform 112. In certain embodiments, the one or more processing devices 114 (e.g., servers) may then access the one or more audio files of patient speech data 118 and analyze the one or more audio files of patient speech data 118 to quantify one or more speech variables utilizing the one or more audio files of patient speech data 118. For example, in certain embodiments, the one or more processing devices 114 (e.g., servers) may utilize one or more machine-learning models (e.g., a natural-language processing (NLP) model, a transformer-based language model, an automatic speech recognition (ASR) model) to convert raw audio files of patient speech data 118 into a textual representation (e.g., transcript) or other representational data to determine, for example, one or more linguistic speech variables and/or one or more acoustic speech variables. For example, in some embodiments, the one or more linguistic speech variables and/or one or more acoustic speech variables may include a filled-pause speech variable, a word frequency speech variable, a vocabulary diversity speech variable, and so forth.

For example, in certain embodiments, the one or more processing devices 114 (e.g., servers) may analyze the one or more audio files of patient speech data 118 to quantify the filled-pause speech variable by analyzing the one or more audio files of patient speech data 118 to identify pauses between words in the one or more audio files of patient speech data 118. In certain embodiments, the one or more processing devices 114 (e.g., servers) may identify pauses between words by first identifying the pauses that qualify as filled pauses. For example,

in some embodiments, the one or more processing devices 114 (e.g., servers) may identify the pauses that qualify as filled pauses by measuring, for example, excessive usage of verbal fillers (e.g., “Um”, “Ah”, “Er”, “Uh”, “You know”, “Like”, “I mean”, “Kinda”, “Hmm”, “Kinda like”) between spoken words. In another embodiment, the one or more processing devices 114 (e.g., servers) may identify the pauses that qualify as filled pauses by measuring, for example, extensive silence between spoken words (e.g., a silence period of more than 2 seconds, more than 3 seconds, more than 4 seconds, or more than 5 seconds).

In certain embodiments, the one or more processing devices 114 (e.g., servers) may then compute a sum of a total number of the pauses between words in the transcript of the audio files of patient speech data 118 and a total number of the words in the transcript of the audio files of patient speech data 118, and divide a number of the filled pauses by the sum. In certain embodiments, the one or more processing devices 114 (e.g., servers) may analyze the one or more audio files of patient speech data 118 to quantify the word frequency speech variable. For example, in certain embodiments, the one or more processing devices 114 (e.g., servers) may identify the word frequency speech variable by computing. for each of the words in the transcript of the audio files of patient speech data 118, a frequency score and calculating an average frequency score for the transcript of the audio files of patient speech data 118 utilizing the calculated frequency score.

In certain embodiments, the one or more processing devices 114 (e.g., servers) may analyze the one or more audio files of patient speech data 118 to quantify the vocabulary diversity speech variable. For example, in certain embodiments, for each of a number of windows in the transcript of the audio files of patient speech data 118, the one or more processing devices 114 (e.g., servers) may compute a number of unique words in the window, compute a total number of words in the window, and compute a vocabulary diversity score for the window by dividing the number of unique words by the total number of words. In one embodiment, each window may include, for example, an equal number of contiguous words in the transcript of the audio files of patient speech data 118. In certain embodiments, the one or more processing devices 114 (e.g., servers) may then calculate an average vocabulary diversity score for the transcript of the audio files of patient speech data 118 utilizing the computed vocabulary diversity scores for each of the windows in the transcript of the audio files of patient speech data 118.

In certain embodiments, upon quantifying the filled-pause speech variable, the word frequency speech variable, the vocabulary diversity speech variable, and so forth, the one or more processing devices 114 (e.g., servers) may then correlate one or more of the quantified filled-pause speech variable. the word frequency speech variable. and the vocabulary diversity speech variable with a standardized uptake value ratio (SUVR) of one or more specific Tau positron emission tomography (PET) tracers known to indicate severity and progression of AD to generate an estimate of Tau accumulation 120 for the one of the number of patients 102A, 102B, 102C, and 102D for which the transcript of the audio files of patient speech data 118 corresponds.

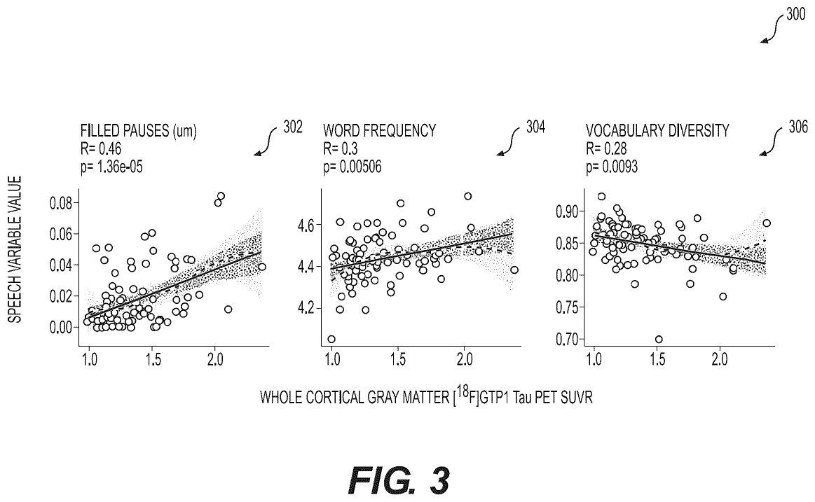

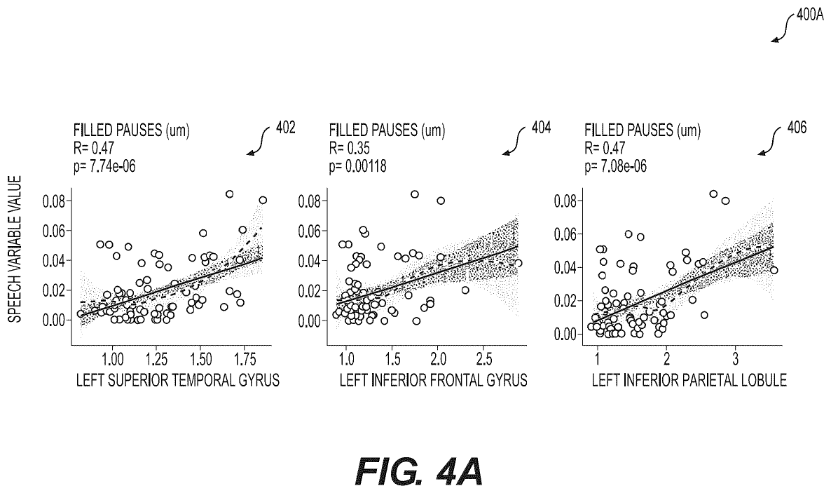

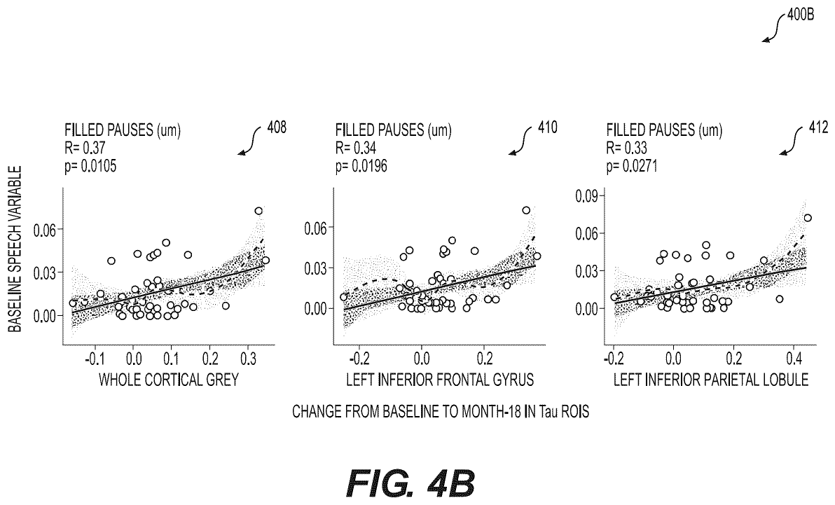

For example, as will be further described with respect to FIGS. 3, 4A, 4B, the one or more processing devices 114 (e.g., servers) may perform one or more Pearson's correlations, for example, between 1) a mapping of one or more measures of uptake (e.g., SUVR) of a Tau PET tracer representing Tau present in brains of other patients to quantified speech variables of the other patients and 2) one or more of the quantified filled-pause speech variable, the word frequency speech variable, the vocabulary diversity speech variable. For example, in certain embodiments, the one or more measures of uptake of the Tau PET tracer may include one or more measures of uptake (e.g., SUVR) of a [¹⁸F]GTP1 Tau PET tracer, an F-18 flortaucipir Tau PET tracer, a (18)F-THK5351 Tau PET tracer, a [¹⁸F]MK-6240) Tau PET tracer, a RO-948 Tau PET tracer, a PI-2014 Tau PET tracer, a PI-2620 Tau PET tracer, or a T-808 Tau PET tracer.

In certain embodiments, utilizing the mapping and one or more of the quantified filled-pause speech variable, the word frequency speech variable, the vocabulary diversity speech variable for the corresponding one of the number of patients 102A, 102B, 102C, and 102D, the one or more processing devices 114 (e.g., servers) may estimate an amount of uptake (e.g., SUVR) of a PET tracer by Tau predicted to be present in a brain of the corresponding one of the number of patients 102A, 102B, 102C, and 102D. Particularly, in some embodiments, one or more specific Tau PET tracers may provide biomarkers that indicate severity and progression of AD. Thus. in accordance with the presently disclosed embodiments, by correlating one or more of the quantified filled-pause speech variable, the word frequency speech variable, the vocabulary diversity speech variable for the corresponding one of the number of patients 102A, 102B, 102C, and 102D to the SUVR values of the one or more specific Tau PET tracers known to indicate severity and progression of AD, the one or more processing devices 114 (e.g., servers) may determine an estimate of Tau accumulation based on one or more of the quantified filled-pause speech variable, the word frequency speech variable, the vocabulary diversity speech variable for the corresponding one of the number of patients 102A, 102B, 102C, and 102D, and thereby detect severity and progression of AD in the corresponding one of the number of patients 102A, 102B, 102C, and 102D.

Indeed, while the present embodiments may be discussed primarily with respect to detecting severity and progression of AD tauopathy, it should be appreciated that the present embodiments of determining an estimate of Tau accumulation based on one or more of the quantified filled-pause speech variable, the word frequency speech variable, the vocabulary diversity speech variable may also be utilized to detect severity and progression of any of various tauopathies (e.g., neurodegenerative diseases or disorders characterized by the deposition of abnormal tau protein in the brain) including, for example, Pick disease tauopathy, progressive supranuclear palsy (PSP) tauopathy, corticobasal degeneration (CBD) tauopathy, argyrophilic grain disease (AGD) tauopathy, globular glial tauopathy, primary age-related tauopathy (PART), neurofibrillary tangle predominant dementia (NFTPD) tauopathy, chronic traumatic encephalopathy (CTE) tauopathy. Parkinson's disease (PD) tauopathy, or aging-related tau astrogliopathy (ARTAG) tauopathy, and so forth.

In certain embodiments, as part of generating the estimate of Tau accumulation 120 (and/or estimate of progression of AD 120), the one or more processing devices 114 (e.g., servers) may determine a rate of progression of AD by measuring a change in one or more of the quantified filled-pause speech variable, the word frequency speech variable, the vocabulary diversity speech variable for the corresponding one of the number of patients 102A, 102B, 102C, and 102D over a period of time. For example, in some embodiments, utilizing the determined rate of progression of AD, the one or more processing devices 114 (e.g., servers) may predict a change in performance of the corresponding one of the number of patients 102A, 102B, 102C, and 102D on a clinical assessment selected, for example, from a set of clinical assessments including a Mini Mental State Examination (MMSE), a Clinical Dementia Rating (CDR) Scale interview, a Clinical Dementia Rating—Sum of Boxes (CDR-SB) interview, a Alzheimer's Disease Assessment Scale—Cognitive Subscale (ADAS-Cog) battery of tests, an Alzheimer's disease Cooperative Study Group—Activities of Daily Living Inventory, a Neuropsychiatric Inventory, a Caregiver Global Impression Scale for Alzheimer's Disease, an Instrumental Activities of Daily Living Scale, and an Amsterdam Activities of Daily Living Questionnaire.

In certain embodiments, as further illustrated by FIG. 1, the one or more processing devices 114 (e.g., servers) may then transmit the generated estimate of Tau accumulation 120 (and/or estimate of progression of AD 120) to a computing device 122 and present a notification or report 124 to a clinician 126 that may be associated with the corresponding one of the number of patients 102A, 102B, 102C, and 102D. In one embodiment, the one or more processing devices 114 (e.g., servers) may also transmit the generated estimate of Tau accumulation 120 (and/or estimate of progression of AD 120) to the corresponding one of the number of patients 102A, 102B, 102C, and 102D via the respective electronic device 104A, 104B, 104C, or 104D. In certain embodiments, the clinician 126 may examine the notification or report 124 and communicate with the corresponding one of the number of patients 102A, 102B, 102C, and 102D via the respective telehealth application 106A (e.g., “Telehealth App 1”), 106B (e.g., “Telehealth App 2”), 106C (e.g., “Telehealth App 3”), or 106D (e.g., “Telehealth App N”) regarding the cognitive health of the corresponding one of the number of patients 102A, 102B, 102C, and 102D.

For example, in certain embodiments, based on a medical review and analysis of the generated estimate of Tau accumulation 120 (and/or estimate of progression of AD 120), the clinician 126 may communicate via the computing device 122 a recommendation for administration of a treatment regimen or therapeutic regimen for the corresponding one of the number of patients 102A, 102B, 102C, and 102D. In response to receiving the input of the clinician 126 via the computing device 122. the one or more processing devices 114 (e.g., servers) may generate a recommendation for administration of a therapeutic agent consisting of at least one compound selected, for example, from a set including compounds against oxidative stress, anti-apoptotic compounds, metal chelators, inhibitors of DNA repair, 3-amino-1-propanesulfonic acid (3APS), 1,3-propanedisulfonate (1,3PDS), secretase activators, beta-and gamma-secretase inhibitors, tau proteins, anti-Tau antibodies, anti-Tau agents, neurotransmitters, beta-sheet breakers, anti-inflammatory molecules, an atypical antipsychotic, a cholinesterase inhibitor, other drugs, and nutritive supplements, a therapeutic agent selected from a set including: a symptomatic medication, a neurological drug, a corticosteroid, an antibiotic, an antiviral agent, an anti-Tau antibody, a Tau inhibitor, an anti-amyloid beta antibody, an beta-amyloid aggregation inhibitor, a therapeutic agent that binds to a target, an anti-BACE1 antibody, a BACE1 inhibitor, a cholinesterase inhibitor, an NMDA receptor antagonist, a monoamine depletory, an ergoloid mesylate, an anticholinergic antiparkinsonism agent, a dopaminergic antiparkinsonism agent, a tetrabenazine, an anti-inflammatory agent, a hormone, a vitamin, a dimebolin, a homotaurine, a serotonin receptor activity modulator, an interferon, and a glucocorticoid. In certain embodiments, the one or more processing devices 114 (e.g., servers) may then transmit a notification regarding the recommendation for administration of a treatment regimen or therapeutic regimen for the corresponding one of the number of patients 102A, 102B, 102C, and 102D respective via the respective electronic device 104A, 104B, 104C, or 104D.

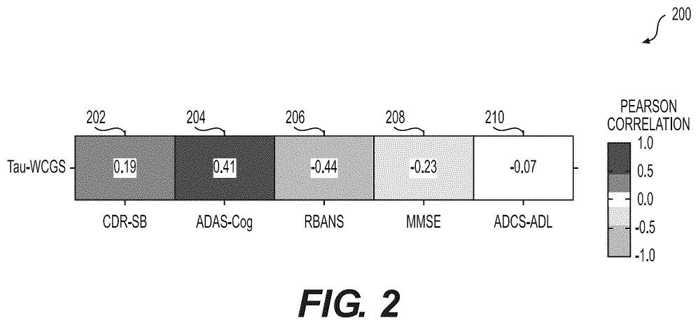

FIG. 2 illustrates a table diagram 200 of cross-sectional correlations between whole cortical grey [¹⁸F]GTP1 SUVR and global clinical scores, in accordance with the presently disclosed embodiments. As depicted. the table diagram 200 illustrates Pearson's correlation between a Tau worst-case gain sensitivity (Tau-WCGS) cognitive score 201 respectively correlated with a CDR-SB global cognitive score 202 (e.g., “0.19”), an ADAS-Cog global cognitive score 204 (e.g., “0.41”), a Repeatable Battery for the Assessment of Neuropsychological Status (RBANS) global cognitive score 206 (e.g., “−0.44”), a Mini Mental State Examination (MMSE) global cognitive score 208 (e.g., “−0.23”), and an Alzheimer's Disease Cooperative Study—Activities of Daily Living Scale (ADCS-ADL) global cognitive score 210 (e.g., “−0.07”). Specifically, in certain embodiments, the global cognitive scores, 202, 204, 206, 208, and 210 may represent, for example, Pearson's correlation coefficients (e.g., one or more numbers between −1 and +1 that reflect the propensity for two random variables to have a linear association) and may illustrate generally that Tau accumulation represented by Tau-WCGS) cognitive score 201 correlates with severity and progression of AD.

FIG. 3 illustrates a plot diagrams 300 of cross-sectional correlations between whole cortical grey [¹⁸F]GTP1 SUVR and speech variables. in accordance with the presently disclosed embodiments. In one embodiment, the cross-sectional correlations of plot diagrams 300 were generated based on patient speech data and PET scan data collected from one or more patients. As depicted, in accordance with the presently disclosed embodiments, the plot diagrams 300 illustrate a plot diagram 302 of a Pearson's correlation between the SUVR of Tau PET tracer [¹⁸F]GTP1 and a quantified filled-pause speech variable, a plot diagram 304 of a Pearson's correlation between the SUVR of Tau PET tracer [¹⁸F]GTP1 and a quantified word-frequency speech variable. and plot diagram 306 of a Pearson's correlation between the SUVR of Tau PET tracer [¹⁸F]GTP1 and a quantified vocabulary-diversity speech variable. Specifically, in certain embodiments, the plot diagrams 302, 304, and 306 illustrate the Pearson's correlation coefficients (e.g., “R” representing a number between −1 and +1 that reflect the propensity for two random variables to have a linear association) for the quantified filled-pause speech variable (e.g., R=0.46), the quantified word-frequency speech variable (e.g., R=0.3), and the quantified vocabulary-diversity speech variable (e.g., R=−0.28), respectively. Thus, the plot diagrams 302, 304, and 306 illustrate that Tau accumulation correlates with the quantified filled-pause speech variable, the quantified word-frequency speech variable, and the quantified vocabulary-diversity speech variable, respectively, with the quantified filled-pause speech variable having the strongest correlation with Tau accumulation as compared to the quantified word-frequency speech variable and the quantified vocabulary-diversity speech variable.

FIG. 4A illustrates a plot diagrams 400A of cross-sectional correlations between [¹⁸F]GTP1 SUVR and filled-pauses generalize across regions of interests (ROIs) of a patient's brain, in accordance with the presently disclosed embodiments. In one embodiment, the cross-sectional correlations of plot diagrams 400A were generated based on patient speech data and PET scan data collected from one or more patients. As depicted, the plot diagrams 400A illustrate a plot diagram 402 of a Pearson's correlation between the SUVR of Tau PET tracer [¹⁸F]GTP1 and a quantified filled-pause speech variable for the left superior temporal gyrus of the patient's brain, a plot diagram 404 of a Pearson's correlation between the SUVR of Tau PET tracer [¹⁸F]GTP1 and a quantified filled-pause speech variable for the left inferior frontal gyrus of the patient's brain, and plot diagram 406 of a Pearson's correlation between the SUVR of Tau PET tracer [¹⁸F]GTP1 and a quantified filled-pause speech variable for the left inferior parietal lobule of the patient's brain. Specifically, in certain embodiments, the plot diagrams 402, 404, and 406 illustrate the Pearson's correlation coefficients for the quantified filled-pause speech variable for the left superior temporal gyrus of the patient's brain (e.g., R=0.47), the quantified filled-pause speech variable for the left inferior frontal gyrus of the patient's brain (e.g., R=0.35), and the quantified filled-pause speech variable for the left inferior parietal lobule of the patient's brain (e.g., R=0.47), respectively, with the quantified filled-pause speech variables for the left superior temporal gyrus of the patient's brain and the left inferior parietal lobule of the patient's brain having the strongest correlations with the SUVR of Tau PET tracer [¹⁸F]GTP1.

FIG. 4B illustrates a plot diagrams 400B of baseline filled-pauses correlated with increases in [¹⁸F]GTP1 SUVR over 18 months, in accordance with the presently disclosed embodiments. In one embodiment, the correlations of plot diagrams 400B were generated based on patient speech data and PET scan data collected from one or more patients over 18 months. As depicted, the plot diagrams 400B illustrate a plot diagram 408 of a Pearson's correlation between the baseline filled-pause and increases in [¹⁸F]GTP1 SUVR over 18 months for the whole cortical grey of the patient's brain, a plot diagram 410 of a Pearson's correlation between the baseline filled-pause and increases in [¹⁸F]GTP1 SUVR over 18 months for the left inferior frontal gyrus of the patient's brain, and plot diagram 412 of a Pearson's correlation between the baseline filled-pause and increases in [¹⁸F]GTP1 SUVR over 18 months for the left inferior parietal lobule of the patient's brain. Specifically, in certain embodiments, the plot diagrams 408, 410, and 412 illustrate the Pearson's correlation coefficients for the quantified filled-pause speech variable for the left superior temporal gyrus of the patient's brain (e.g., R=0.37), the quantified filled-pause speech variable for the left inferior frontal gyrus of the patient's brain (e.g., R=0.34), and the quantified filled-pause speech variable for the left inferior parietal lobule (e.g., R=0.33), respectively, with the baseline filled-pause for the whole cortical grey of the patient's brain having the strongest correlation with Tau accumulation over 18 months.

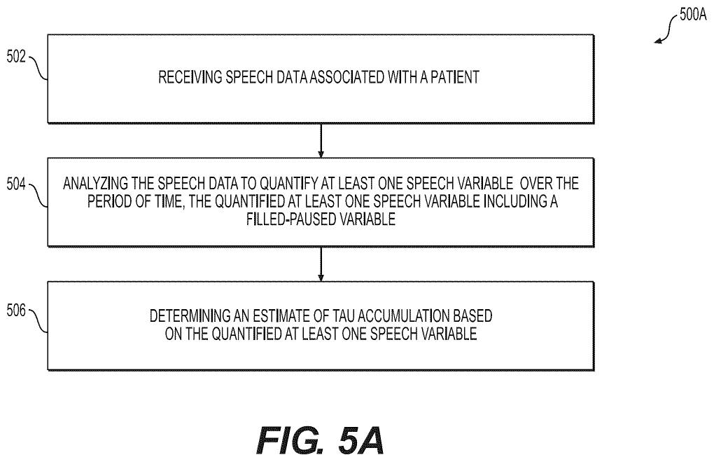

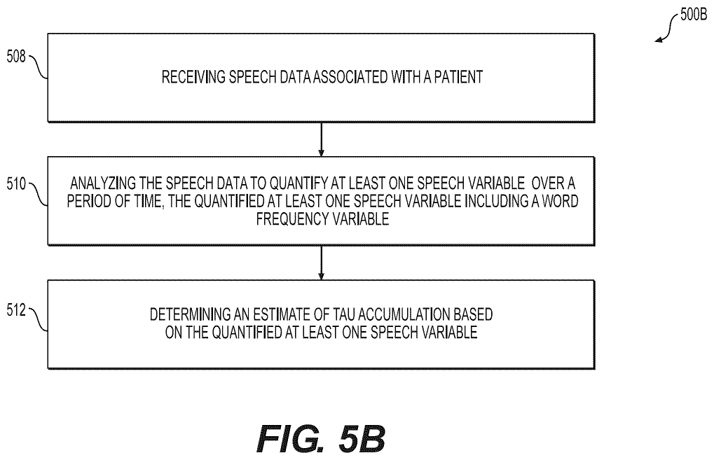

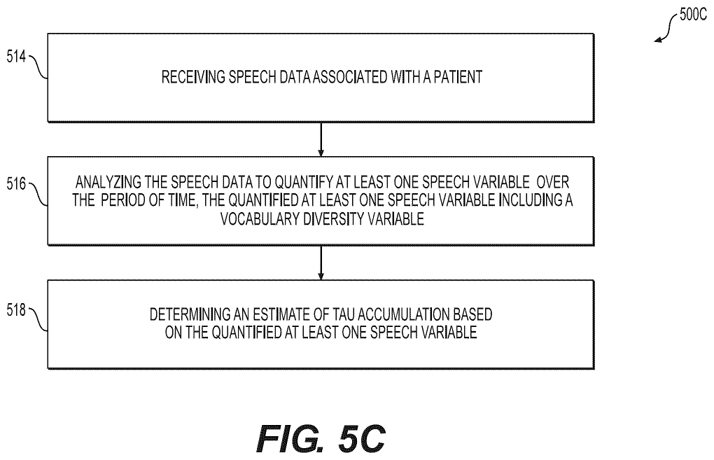

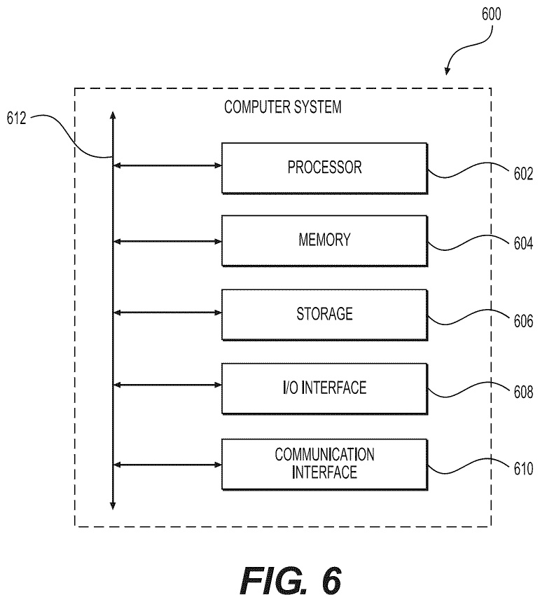

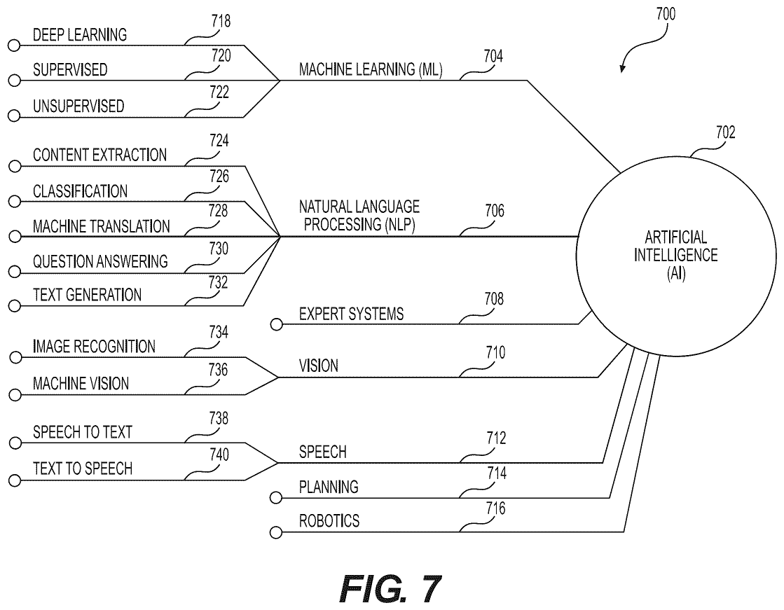

FIG. 5A illustrates a flow diagram 500A for determining an estimate of Tau accumulation in a patient based on at least a filled-pause quantified speech variable and detecting severity and progression of neurodegenerative disease in the patient, in accordance with the presently disclosed embodiments. The flow diagram 500A may be performed utilizing one or more processing devices (e.g., computing system and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 500A may begin at block 502 with one or more processing devices receiving speech data associated with a patient. The flow diagram 500A may then continue at block 504 with one or more processing devices analyzing the speech data to quantify at least one speech variable over a period of time, in which the at least one speech variable includes a filled-pause variable. The flow diagram 500A may then conclude at block 506 with one or more processing devices determining an estimate of Tau accumulation based on the quantified at least one speech variable.

FIG. 5B illustrates a flow diagram 500B for determining an estimate of Tau accumulation in a patient based on at least a word frequency quantified speech variable and detecting severity and progression of neurodegenerative disease in the patient, in accordance with the presently disclosed embodiments. The flow diagram 500B may be performed utilizing one or more processing devices (e.g., computing system and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 500B may begin at block 508 with one or more processing devices receiving speech data associated with a patient. The flow diagram 500B may then continue at block 510 with one or more processing devices analyzing the speech data to quantify at least one speech variable over a period of time, in which the at least one speech variable includes a word frequency variable. The flow diagram 500B may then conclude at block 512 with one or more processing devices determining an estimate of Tau accumulation based on the quantified at least one speech variable.

FIG. 5C illustrates a flow diagram 500C for determining an estimate of Tau accumulation in a patient based on at least a vocabulary diversity quantified speech variable and detecting severity and progression of neurodegenerative disease in the patient, in accordance with the presently disclosed embodiments. The flow diagram 500C may be performed utilizing one or more processing devices (e.g., computing system and artificial intelligence architecture to be discussed below with respect to FIGS. 6 and 7) that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various medical profile data and making one or more decisions based thereon), software (e.g., instructions running/executing on one or more processors), firmware (e.g., microcode), or some combination thereof.

The flow diagram 500C may begin at block 514 with one or more processing devices receiving speech data associated with a patient. The flow diagram 500C may then continue at block 516 with one or more processing devices analyzing the speech data to quantify at least one speech variable over a period of time, in which the at least one speech variable includes a vocabulary diversity variable. The flow diagram 500C may then conclude at block 518 with one or more processing devices determining an estimate of Tau accumulation based on the quantified at least one speech variable.

Accordingly, as generally set forth by the flow diagram 500A of FIG. 5A, the flow diagram 500B of FIG. 5B, and the flow diagram 500C of FIG. 5C, the present embodiments are directed toward one or more computing devices, methods, and non-transitory computer-readable media that may be utilized to determine an estimate of Tau accumulation in a patient based on at least a filled-pause quantified speech variable and to detect severity and progression of neurodegenerative disease in the patient. Specifically, in accordance with the presently disclosed embodiments, one or more computing devices may utilize one or more machine-learning models (e.g., a natural-language processing (NLP) model, a transformer-based language model, an automatic speech recognition (ASR) model) to convert raw audio files of patient speech data into a textual transcript and analyze one or more linguistic speech variables and/or one or more acoustic speech variables for determining an estimate of Tau accumulation for a patient to which the patient speech data corresponds. For example, in some embodiments, the one or more computing devices may analyze the textual transcript to calculate one or more of a filled-pause speech variable, a word-frequency speech variable, a vocabulary-diversity speech variable, and so forth that may be then correlated with one or more the SUVR values of one or more specific Tau PET tracers known to indicate severity and progression of AD. Thus, the one or more processing devices may determine an estimate of Tau accumulation based on one or more of the quantified filled-pause speech variable, the word frequency speech variable, the vocabulary diversity speech variable for the corresponding one of the number of patients, and may thereby detect severity and progression of AD in the patient.

FIG. 6 illustrates an example computing system 600 that may be utilized to determine an estimate of Tau accumulation in a patient based on at least a filled-pause quantified speech variable and to detect severity and progression of neurodegenerative disease in the patient. in accordance with the presently disclosed embodiments. In certain embodiments, the computing system 600 may perform one or more steps of one or more methods described or illustrated herein. In certain embodiments, the computing system 600 provide functionality described or illustrated herein. In certain embodiments, software running on the computing system 600 performs one or more steps of one or more methods described or illustrated herein or provides functionality described or illustrated herein. Certain embodiments include one or more portions of the computing systems 600. Herein, reference to a computer system may encompass a computing device, and vice versa, where appropriate. Moreover, reference to a computer system may encompass one or more computer systems, where appropriate.

This disclosure contemplates any suitable number of computing systems 600. This disclosure contemplates computing system 600 taking any suitable physical form. As example and not by way of limitation, computing system 600 may be an embedded computer system, a system-on-chip (SOC), a single-board computer system (SBC) (e.g., a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, the computing system 600 may include one or more computing systems 600; be unitary or distributed; span multiple locations; span multiple machines; span multiple data centers; or reside in a cloud, which may include one or more cloud components in one or more networks.

Where appropriate, the computing system 600 may perform without substantial spatial or temporal limitation one or more steps of one or more methods described or illustrated herein. As an example, and not by way of limitation, the computing system 600 may perform in real time or in batch mode one or more steps of one or more methods described or illustrated herein. The computing system 600 may perform at different times or at different locations one or more steps of one or more methods described or illustrated herein, where appropriate.

In certain embodiments, the computing system 600 includes a processor 602, memory 604, storage 606, an input/output (I/O) interface 608, a communication interface 610, and a bus 612. Although this disclosure describes and illustrates a particular computer system having a particular number of particular components in a particular arrangement, this disclosure contemplates any suitable computer system having any suitable number of any suitable components in any suitable arrangement. In certain embodiments, processor 602 includes hardware for executing instructions, such as those making up a computer program. As an example, and not by way of limitation, to execute instructions, processor 602 may retrieve (or fetch) the instructions from an internal register, an internal cache, memory 604, or storage 606; decode and execute them; and then write one or more results to an internal register, an internal cache, memory 604, or storage 606. In certain embodiments, processor 602 may include one or more internal caches for data, instructions, or addresses. This disclosure contemplates processor 602 including any suitable number of any suitable internal caches, where appropriate. As an example, and not by way of limitation, processor 602 may include one or more instruction caches, one or more data caches, and one or more translation lookaside buffers (TLBs). Instructions in the instruction caches may be copies of instructions in memory 604 or storage 606, and the instruction caches may speed up retrieval of those instructions by processor 602.

Data in the data caches may be copies of data in memory 604 or storage 606 for instructions executing at processor 602 to operate on; the results of previous instructions executed at processor 602 for access by subsequent instructions executing at processor 602 or for writing to memory 604 or storage 606; or other suitable data. The data caches may speed up read or write operations by processor 602. The TLBs may speed up virtual-address translation for processor 602. In certain embodiments, processor 602 may include one or more internal registers for data, instructions, or addresses. This disclosure contemplates processor 602 including any suitable number of any suitable internal registers, where appropriate. Where appropriate, processor 602 may include one or more arithmetic logic units (ALUs); be a multi-core processor; or include one or more processors 602. Although this disclosure describes and illustrates a particular processor, this disclosure contemplates any suitable processor.

In certain embodiments, memory 604 includes main memory for storing instructions for processor 602 to execute or data for processor 602 to operate on. As an example, and not by way of limitation, the computing system 600 may load instructions from storage 606 or another source (such as, for example, another computing system 600) to memory 604. Processor 602 may then load the instructions from memory 604 to an internal register or internal cache. To execute the instructions, processor 602 may retrieve the instructions from the internal register or internal cache and decode them. During or after execution of the instructions, processor 602 may write one or more results (which may be intermediate or final results) to the internal register or internal cache. Processor 602 may then write one or more of those results to memory 604.

In certain embodiments, processor 602 executes only instructions in one or more internal registers or internal caches or in memory 604 (as opposed to storage 606 or elsewhere) and operates only on data in one or more internal registers or internal caches or in memory 604 (as opposed to storage 606 or elsewhere). One or more memory buses (which may each include an address bus and a data bus) may couple processor 602 to memory 604. Bus 612 may include one or more memory buses, as described below. In certain embodiments, one or more memory management units (MMUs) reside between processor 602 and memory 604 and facilitate accesses to memory 604 requested by processor 602. In certain embodiments, memory 604 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 604 may include one or more memory devices 604, where appropriate. Although this disclosure describes and illustrates particular memory, this disclosure contemplates any suitable memory.

In certain embodiments, storage 606 includes mass storage for data or instructions. As an example, and not by way of limitation, storage 606 may include a hard disk drive (HDD), a floppy disk drive, flash memory, an optical disc, a magneto-optical disc, magnetic tape, or a Universal Serial Bus (USB) drive or a combination of two or more of these. Storage 606 may include removable or non-removable (or fixed) media, where appropriate. Storage 606 may be internal or external to the computing system 600, where appropriate. In certain embodiments, storage 606 is non-volatile, solid-state memory. In certain embodiments, storage 606 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 606 taking any suitable physical form. Storage 606 may include one or more storage control units facilitating communication between processor 602 and storage 606, where appropriate. Where appropriate, storage 606 may include one or more storages 606. Although this disclosure describes and illustrates particular storage, this disclosure contemplates any suitable storage.

In certain embodiments, I/O interface 608 includes hardware, software, or both, providing one or more interfaces for communication between the computing system 600 and one or more I/O devices. The computing system 600 may include one or more of these I/O devices, where appropriate. One or more of these I/O devices may enable communication between a person and the computing system 600. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 606 for them. Where appropriate, I/O interface 608 may include one or more device or software drivers enabling processor 602 to drive one or more of these I/O devices. I/O interface 608 may include one or more I/O interfaces 606, where appropriate. Although this disclosure describes and illustrates a particular I/O interface, this disclosure contemplates any suitable I/O interface.

In certain embodiments, communication interface 610 includes hardware, software, or both providing one or more interfaces for communication (such as, for example, packet-based communication) between the computing system 600 and one or more other computer systems 600 or one or more networks. As an example, and not by way of limitation, communication interface 610 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 610) for it.

As an example, and not by way of limitation. the computing system 600 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the computing system 600 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. The computing system 600 may include any suitable communication interface 610 for any of these networks, where appropriate. Communication interface 610 may include one or more communication interfaces 610, where appropriate. Although this disclosure describes and illustrates a particular communication interface, this disclosure contemplates any suitable communication interface.

In certain embodiments, bus 612 includes hardware, software, or both coupling components of the computing system 600 to each other. As an example, and not by way of limitation, bus 612 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 612 may include one or more buses 612, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

FIG. 7 illustrates a diagram 700 of an example artificial intelligence (AI) architecture 702 (which may be included as part of the computing system 600 as discussed above with respect to FIG. 6) that may be utilized to determine an estimate of Tau accumulation in a patient based on at least a filled-pause quantified speech variable and to detect severity and progression of neurodegenerative disease in the patient, in accordance with the presently disclosed embodiments. In certain embodiments, the AI architecture 702 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), and/or other processing device(s) that may be suitable for processing various medical profile data and making one or more decisions based thereon), software (e.g., instructions running/executing on one or more processing devices), firmware (e.g., microcode), or some combination thereof.

In certain embodiments, as depicted by FIG. 7, the AI architecture 702 may include machine learning (ML) algorithms and functions 704, natural language processing (NLP) algorithms and functions 706, expert systems 708, computer-based vision algorithms and functions 710, speech recognition algorithms and functions 712, planning algorithms and functions 714, and robotics algorithms and functions 716. In certain embodiments, the ML algorithms and functions 704 may include any statistics-based algorithms that may be suitable for finding patterns across large amounts of data (e.g., “Big Data” such as genomics data. proteomics data, metabolomics data, metagenomics data, transcriptomics data, medication data, medical diagnostics data, medical procedures data, medical diagnoses data, medical symptoms data, demographics data, patient lifestyle data, physical activity data, family history data, socioeconomics data, geographic environment data, and so forth). For example, in certain embodiments, the ML algorithms and functions 704 may include deep learning algorithms 718, supervised learning algorithms 720, and unsupervised learning algorithms 722.

In certain embodiments, the deep learning algorithms 718 may include any artificial neural networks (ANNs) that may be utilized to learn deep levels of representations and abstractions from large amounts of data. For example, the deep learning algorithms 718 may include ANNs, such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 720 may include any algorithms that may be utilized to apply, for example, what has been learned in the past to new data using labeled examples for predicting future events. For example, starting from the analysis of a known training data set, the supervised learning algorithms 720 may produce an inferred function to make predictions about the output values. The supervised learning algorithms 600 may also compare its output with the correct and intended output and find errors in order to modify the supervised learning algorithms 720 accordingly. On the other hand, the unsupervised learning algorithms 722 may include any algorithms that may applied, for example, when the data used to train the unsupervised learning algorithms 722 are neither classified nor labeled. For example, the unsupervised learning algorithms 722 may study and analyze how systems may infer a function to describe a hidden structure from unlabeled data.

In certain embodiments, the NLP algorithms and functions 706 may include any algorithms or functions that may be suitable for automatically manipulating natural language, such as speech and/or text. For example, in some embodiments, the NLP algorithms and functions 706 may include content extraction algorithms or functions 724, classification algorithms or functions 726, machine translation algorithms or functions 728, question answering (QA) algorithms or functions 730, and text generation algorithms or functions 732. In certain embodiments, the content extraction algorithms or functions 724 may include a means for extracting text or images from electronic documents (e.g., webpages, text editor documents, and so forth) to be utilized, for example, in other applications.

In certain embodiments, the classification algorithms or functions 726 may include any algorithms that may utilize a supervised learning model (e.g., logistic regression, naïve Bayes. stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 728 may include any algorithms or functions that may be suitable for automatically converting source text in one language, for example, into text in another language. The QA algorithms or functions 730 may include any algorithms or functions that may be suitable for automatically answering questions posed by humans in, for example, a natural language, such as that performed by voice-controlled personal assistant devices. The text generation algorithms or functions 732 may include any algorithms or functions that may be suitable for automatically generating natural language texts.

In certain embodiments, the expert systems 708 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 710 may include any algorithms or functions that may be suitable for automatically extracting information from images (e.g., photo images, video images). For example, the computer-based vision algorithms and functions 710 may include image recognition algorithms 734 and machine vision algorithms 736. The image recognition algorithms 734 may include any algorithms that may be suitable for automatically identifying and/or classifying objects, places, people, and so forth that may be included in, for example, one or more image frames or other displayed data. The machine vision algorithms 736 may include any algorithms that may be suitable for allowing computers to “see”, or, for example, to rely on image sensors cameras with specialized optics to acquire images for processing, analyzing, and/or measuring various data characteristics for decision making purposes.

In certain embodiments, the speech recognition algorithms and functions 712 may include any algorithms or functions that may be suitable for recognizing and translating spoken language into text, such as through automatic speech recognition (ASR), computer speech recognition, speech-to-text (STT) 738, or text-to-speech (TTS) 740 in order for the computing to communicate via speech with one or more users, for example. In certain embodiments, the planning algorithms and functions 714 may include any algorithms or functions that may be suitable for generating a sequence of actions, in which each action may include its own set of preconditions to be satisfied before performing the action. Examples of AI planning may include classical planning, reduction to other problems, temporal planning, probabilistic planning, preference-based planning, conditional planning, and so forth. Lastly, the robotics algorithms and functions 716 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method, a storage medium, a system and a computer program product, wherein any feature mentioned in one claim category, e.g. method, may be claimed in another claim category, e.g. system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

What is claimed is:

1. A method, comprising, by one or more computing devices:
receiving speech data associated with a patient;
analyzing the speech data to quantify at least one speech variable over a period of time, wherein the at least one speech variable comprises a filled-pause variable; and
determining an estimate of Tau accumulation based on the quantified at least one speech variable.

2. The method of claim 1, wherein receiving the speech data comprises receiving an audio file comprising an electronic recording of the patient speaking.

3. The method of claim 2, wherein analyzing the speech data to quantify at least one speech variable comprises:
analyzing the audio file to quantify the at least one speech variable, wherein the at least one speech variable comprises an acoustic speech variable.

4. The method of claim 3, wherein analyzing the audio file to quantify the filled-pause variable comprises:
analyzing the audio file to identify pauses between words in the speech data;
identifying the pauses that qualify as filled pauses;
computing a sum of a total number of the pauses between words in the transcript and a total number of the words in the transcript;
dividing a number of the filled pauses by the sum; and
returning the quantified filled-pause variable.

5. The method of claim 1, wherein determining the estimate of Tau accumulation comprises:
mapping one or more measures of uptake of a positron emission tomography (PET) tracer by Tau present in brains of other subjects to quantified speech variables of the other subjects; and
estimating, based on the mapping and the one or more quantified speech variables of the patient, an amount of uptake of a PET tracer by Tau predicted to be present in a brain of the patient.

6. The method of claim 1, further comprising: classifying the patient, based on the estimate of Tau accumulation, as having a neurodegenerative disease.

7. The method of claim 1, further comprising transmitting a notification regarding the estimate of Tau accumulation to a computing device associated with a clinician and/or the patient.

8. The method of claim 1, further comprising, in response to determining the estimate of Tau accumulation, generating a recommendation for administration of a therapeutic agent consisting of at least one compound selected from a group consisting of compounds against oxidative stress, anti-apoptotic compounds, metal chelators, inhibitors of DNA repair, 3-amino-1-propanesulfonic acid (3APS), 1,3-propanedisulfonate (1,3PDS), secretase activators, beta-and gamma-secretase inhibitors, tau proteins, anti-Tau antibodies, anti-Tau agents, gene therapies, neurotransmitters, beta-sheet breakers, anti-inflammatory molecules, an atypical antipsychotic, a cholinesterase inhibitor, other drugs, and nutritive supplements, a therapeutic agent selected from the group consisting of: a symptomatic medication, a neurological drug, a corticosteroid, an antibiotic, an antiviral agent, an anti-Tau antibody, a Tau inhibitor, an anti-amyloid beta antibody, an beta-amyloid aggregation inhibitor, a therapeutic agent that binds to a target, an anti-BACE1 antibody, a BACE1 inhibitor, a cholinesterase inhibitor, an NMDA receptor antagonist, a monoamine depletory, an ergoloid mesylate, an anticholinergic antiparkinsonism agent, a dopaminergic antiparkinsonism agent, a tetrabenazine, an anti-inflammatory agent, a hormone, a vitamin, a dimebolin, a homotaurine, a serotonin receptor activity modulator, an interferon, and a glucocorticoid.

9. A system including one or more computing devices, comprising:
one or more non-transitory computer-readable storage media including instructions; and
one or more processors coupled to the one or more storage media, the one or more processors configured to execute the instructions to:
receive speech data associated with a patient;
analyze the speech data to quantify at least one speech variable over a period of time, wherein the at least one speech variable comprises a filled-pause variable; and
determine an estimate of Tau accumulation based on the quantified at least one speech variable.

10. The system of claim 9, wherein the instructions to receive the speech data further comprise instructions to receive an audio file comprising an electronic recording of the patient speaking.

11. The system of claim 10, wherein the instructions to analyze the speech data to quantify at least one speech variable further comprise instructions to:
analyze the audio file to quantify the at least one speech variable, wherein the at least one speech variable comprises an acoustic speech variable.

12. The system of claim 11, wherein the instructions to analyze the audio file to quantify the filled-pause variable further comprise instructions to:
analyze the audio file to identify pauses between words in the speech data;
identify the pauses that qualify as filled pauses;
compute a sum of a total number of the pauses between words in the transcript and a total number of the words in the transcript;
divide a number of the filled pauses by the sum; and
return the quantified filled-pause variable.

13. The system of claim 9, wherein the instructions to determine the estimate of Tau accumulation further comprise instructions to:
map one or more measures of uptake of a positron emission tomography (PET) tracer by Tau present in brains of other subjects to quantified speech variables of the other subjects; and
estimate, based on the mapping and the one or more quantified speech variables of the patient, an amount of uptake of a PET tracer by Tau predicted to be present in a brain of the patient.

14. The system of claim 9, wherein the instructions further comprise instructions to classify the patient, based on the estimate of Tau accumulation, as having a neurodegenerative disease.

15. The system of claim 9, wherein the instructions further comprise instructions to transmit a notification regarding the estimate of Tau accumulation to a computing device associated with a clinician and/or the patient.

16. The system of claim 9, wherein the instructions further comprise instructions to:
in response to determining the estimate of Tau accumulation, generate a recommendation for administration of a therapeutic agent consisting of at least one compound selected from a group consisting of compounds against oxidative stress, anti-apoptotic compounds, metal chelators, inhibitors of DNA repair, 3-amino-1-propanesulfonic acid (3APS), 1,3-propanedisulfonate (1,3PDS), secretase activators, beta-and gamma-secretase inhibitors, tau proteins, anti-Tau antibodies, anti-Tau agents, gene therapies, neurotransmitters, beta-sheet breakers, anti-inflammatory molecules, an atypical antipsychotic, a cholinesterase inhibitor, other drugs, and nutritive supplements, a therapeutic agent selected from the group consisting of: a symptomatic medication, a neurological drug, a corticosteroid, an antibiotic, an antiviral agent, an anti-Tau antibody, a Tau inhibitor, an anti-amyloid beta antibody, an beta-amyloid aggregation inhibitor, a therapeutic agent that binds to a target, an anti-BACE1 antibody, a BACE1 inhibitor, a cholinesterase inhibitor, an NMDA receptor antagonist, a monoamine depletory, an ergoloid mesylate, an anticholinergic antiparkinsonism agent, a dopaminergic antiparkinsonism agent, a tetrabenazine, an anti-inflammatory agent, a hormone, a vitamin, a dimebolin, a homotaurine, a serotonin receptor activity modulator, an interferon, and a glucocorticoid.

17. A method, comprising, by one or more computing devices:
receiving speech data associated with a patient;
analyzing the speech data to quantify at least one speech variable over a period of time, wherein the at least one speech variable comprises a word frequency variable; and
determining an estimate of Tau accumulation based on the quantified at least one speech variable.

18. A system including one or more computing devices, comprising:
one or more non-transitory computer-readable storage media including instructions; and
one or more processors coupled to the one or more storage media, the one or more processors configured to execute the instructions to:
receive speech data associated with a patient;
analyze the speech data to quantify at least one speech variable over a period of time, wherein the at least one speech variable comprises a word frequency variable; and
determine an estimate of Tau accumulation based on the quantified at least one speech variable.

19. A method, comprising, by one or more computing devices:
receiving speech data associated with a patient;
analyzing the speech data to quantify at least one speech variable over a period of time, wherein the at least one speech variable comprises a vocabulary diversity variable; and
determining an estimate of Tau accumulation based on the quantified at least one speech variable.

20. A system including one or more computing devices, comprising:
one or more non-transitory computer-readable storage media including instructions; and
one or more processors coupled to the one or more storage media, the one or more processors configured to execute the instructions to:
receive speech data associated with a patient;
analyze the speech data to quantify at least one speech variable over a period of time, wherein the at least one speech variable comprises a vocabulary diversity variable; and
determine an estimate of Tau accumulation based on the quantified at least one speech variable.
